(19) United States
(12) Patent Application Publication
(10) Pub. No.: US 2008/0269205 A1
(43) Pub. Date: Oct. 30, 2008

(54) METHODS TO PREVENT VERTICAL TRANSMISSION OF INFECTIOUS DISEASES

(75) Inventors: Nicolas G. Loebel, Redmond, WA (US); Roger Andersen, Ladysmith (CA)

Correspondence Address: DOBRUSIN & THENNISCH PC, 29 W LAWRENCE ST, SUITE 210, PONTIAC, MI, 48342, US

(73) Assignee: Ondine International, Ltd.

(21) Appl. No.: 11/877,212

(22) Filed: Oct. 23, 2007

Related U.S. Application Data
(60) Provisional application No. 60/914,638, filed on Apr. 27, 2007.

(51) Int. Cl. A61K 31/5415; A61P 31/12

(52) U.S. Cl. 514/226.2

(57) ABSTRACT

The present invention presents a method of preventing vertical transmission of an infectious disease comprising: (a) applying a photosensitizing composition to host tissues of birth canal of a mother during the intrapartum period; and (b) applying light to the host tissues after the step (a) at a wavelength absorbed by the photosensitizing composition so as to inhibit or eliminate infectious disease microorganisms that come into contact with the host tissues. The infectious disease may be caused by human immunodeficiency virus type 1, hepatitis B virus, hepatitis C virus, Group B Streptococcus, cytomegalovirus, Listeria monocytogenes, Chiamydia trachomatis, Escherichia coli, herpes simplex virus, Epstein-Barr virus, Toxoplasma gondii, human papilloma virus, and Candida.

### CLAIM OF BENEFIT OF FILING DATE

This application claims the benefit of U.S. Provisional Application Ser. No. 60/914,638 titled: Photodisinfection Delivery Devices and Methods filed on Apr. 27, 2007.

### FIELD OF INVENTION

The present invention relates to methods of preventing mother to child transmission of infectious diseases during the intrapartum period, especially infectious diseases caused by human immunodeficiency virus type 1, hepatitis viruses, Group B Streptococcus, cytomegalovirus, Listeria monocytogenes, Chiamydia trachomatis, Escherichia coli, herpes simplex virus, Epstein-Barr virus, Toxoplasma gondii, and Candida.

### BACKGROUND OF THE INVENTION

Tens of millions of individuals have been infected with human immunodeficiency virus type 1 (“HIV-1 ”) worldwide and millions of new cases are identified every year. About 95% of HIV-1 infections are found in developing countries such as sub-Saharan Africa and Southeast Asia. It has been estimated that 570,000 children became infected with HIV-1 during 1999 and 90% of these infections were acquired in utero, during delivery, or through breast-feeding from their HIV-infected mothers. See Michele L. Dreyfuss and Wafaie W. Fawzi, Micronutrients and Vertical Transmission of HIV-1, Am J Clin Nutr 2002: 75:959-70.

Mother to child transmission of infectious diseases, also known vertical transmission, is well established. There is a high risk of vertical transmission of infectious diseases during the intrapartum period due to the presence of the virus in blood and mucus in the birth canal.

For example, a study has shown that a substantial proportion of vertical transmission of HIV-1 occur during the intrapartum period. “Possible mechanisms include transfusion of the mother's blood to the fetus during labor contractions, infection after rupture of membranes, and direct contact of the fetus with infected secretions or blood from the maternal genital tract.” See The Mode of Delivery and The Risk of Vertical Transmission of Human Immunodeficiency Virus Type 1, The New England Journal of Medicine, Vol. 340, No. 13, 977-987, 977 (1999).

Vertical transmission of hepatitis such as hepatitis B virus (“HBV”) and hepatitis C virus (“HCV”) during the intrapartum period is also known and supported by studies. See Caudai, C., Battiata, M., Riccardi, M. P., Toti, M., Bonazza, P., Padula, M. G., Pianese, M., Valensin, P. E. (2003). Vertical Transmission of the Hepatitis C Virus to Infants of Anti-Human Immunodeficiency Virus-Negative Mothers: Molecular Evolution of Hypervariable Region 1 in Prenatal and Perinatal or Postnatal Infections. J. Clin. Microbiol. 41: 3955-3959; Wallis, D. E, Boxall, E. H (1999). Immunisation of infants at risk of perinatal transmission of hepatitis B: retrospective audit of vaccine uptake. BMJ 318: 1112-1113. In addition to HIV-1, HBV and HCV, vertical transmission of other infectious diseases caused by Group B Streptococcus (“GBS”), cytomegalovirus (“CMV”), Listeria monocytogenes, Chlamydia trachomatis, Escherichia coli (“E. coli”), herpes simplex virus (“HSV”), Epstein-Barr virus (“EBV”), Toxoplasma gondii, human papilloma virus (HPV), and Candida has also been documented. See László Maródi, Neonatal Innate Immunity to Infectious Agents, Infect Immun. 2006 Apr.; 74(4): 1999-2006; INTRAUTERINE AND PERINATAL INFECTION, www.nuigalway.ie/bac/student info/Intrauterine Perinatal Infection.html; M C Meyohas, V Marechal, N Desire, J Bouillie, J Frottier and J C Nicolas, Study of mother-to-child Epstein-Barr virus transmission by means of nested PCRs, J. Virol., 10 1996, 6816-6819, Vol 70, No. 10.

### SUMMARY OF THE INVENTION

It is an object of the present invention to prevent vertical transmission of infectious diseases using photodisinfection and/or sonophotodisinfection during the intrapartum period. Photodisinfection and sonophotodisinfection can prevent vertical transmission of these diseases by inhibiting and/or eliminating infectious disease causing microorganisms that come into contact with host tissues of the birth canal such as HIV-1, HBV, HCV, GBS, CMV, Listeria monocytogenes, Chlamydia trachomatis, E. coli, HSV, EBV, Toxoplasma gondii, HPV, Candida, etc.

Photodisinfection is the use of a photosensitizing composition activated by light to inhibit or eliminate infectious disease causing microorganisms in the desired treatment area. When the photosensitizing composition is activated by light, it is believed to release free radicals causing killing of microorganisms.

Sonophotodisinfection is photodisinfection plus the use of sonic energy to inhibit or eliminate infectious disease causing microorganisms in the desired treatment area. It is believed that the application of sonic energy in a fluid (e.g., photosensitizing composition, blood and mucus in the birth canal, etc.) can create acoustic cavitation. Acoustic cavitation involves the nucleation, growth and collapse of gas/vapor filled bubbles in a fluid. Cavitation is believed to effectively kill microorganisms by physical disruption. For example, the mechanical energy in acoustic cavitation can disrupt the microorganisms surrounding it by the violent shear forces produced around the bubbles. Free radicals in a fluid have also been detected as a direct result of acoustic cavitation. These free radicals are believed to kill microorganisms via cell wall disruption and/or lipid peroxidation. It is also believed that the collapse of the bubbles during acoustic cavitation can be accompanied by a simultaneous emission of light (“sonoluminescence”). The light emitted by sonoluminescence is very broadband and may contain ultraviolet light, which can also be directly detrimental to microorganisms. It is also believed that light emitted via sonoluminescence, when applied to a photosensitizing composition in the birth canal, can release more free radicals, causing further killing of microorganisms.

The present invention provides a method of preventing vertical transmission of an infectious disease comprising: (a) applying a photosensitizing composition to host tissues of birth canal of a mother during the intrapartum period; and (b) applying light to the host tissues after the step (a) at a wavelength absorbed by the photosensitizing composition so as to inhibit or eliminate infectious disease microorganisms that come into contact with the host tissues, preferably without causing physiological damage to the host tissues. The present invention optionally includes applying sonic energy to the host tissues after step (b). It is preferred that the applying the steps (a), (b) and optionally (c) are repeated multiple times during the intrapartum period.

The present invention also provides a method of preventing vertical transmission of human immunodeficiency virus type 1 comprising: (a) applying a photosensitizing composition comprising of methylene blue to host tissues of a birth canal of a mother during intrapartum period; (b) applying light to the host tissues after the step (a) at a wavelength from about 650 nm to about 685 nm that is absorbed by the photosensitizing composition so as to inhibit or eliminate human immunodeficiency virus type 1 that comes into contact with the host tissues; and repeating the steps (a) and (b) at least every two hour during the intrapartum period.

The present invention further provides a method of preventing vertical transmission of hepatitis comprising: (a) applying a photosensitizing composition comprising of methylene blue to host tissues of a birth canal of a mother during intrapartum period; (b) applying light to the host tissues after the step (a) at a wavelength from about 660 nm to about 680 nm that is absorbed by the photosensitizing composition so as to inhibit or eliminate at least one type of hepatitis viruses that comes into contact with the host tissues; and repeating the steps (a) and (b) at least every two hour during the intrapartum period.

The present invention provides a vaginal probe comprising: a member having a base portion, an insert portion adapted for insertion into a vagina of a mother and for providing desired illumination pattern for photodisinfection of host tissues of birth canal of the mother, and a pocket adapted for communication with a waveguide wherein the pocket includes a light dispersing section that is adapted for light communication with distal end of the waveguide which is connected to a light source for delivering light to the insert portion, and the member further includes an electronic assembly adapted for providing sonic energy for sonophotodisinfection of host tissues of birth canal of the mother.

### DESCRIPTION OF THE PREFERRED EMBODIMENT

### I. Definitions

The following terms are intended to have the following general meanings as they are used herein:

1. Birth Canal: the path through which a fetus travels in order to be born formed by the cervix, vagina, and vulva of a mother. The host tissues of the birth canal include at least some of the tissues of the cervix, vagina, and vulva that may come into contact with the fetus during the intrapartum period.

2. Intrapartum period: the time period beginning from the onset of labor to the completion of delivery (including cutting of the umbilical cord) of a fetus from the mother's birth canal.

3. Light: light at any wavelengths that can be absorbed by a photosensitizing composition. Such wavelengths include wavelengths selected from the continuous electromagnetic spectrum such as ultraviolet (“UV”), visible, the infrared (near, mid and far), etc. The wavelengths are generally between about 100 nm to 10,000 nm, with exemplary ranges between about 160 nm to 1600 nm, between about 400 nm to about 900 nm, and between about 500 nm to about 850 nm, although the wavelengths may vary depending upon the particular photosensitizing composition used and the light intensity. Depending on the application, the light produced may be a single wavelength or multiple wavelengths. The light may be produced by any suitable art-disclosed light source(s).

4. Light Source: a light emitting device such as laser, light emitting diode (“LEDs”), incandescent source, fluorescent source, or a combination thereof. The output of the light source is preferably adjustable so that the operator can modify the wavelength, the power output, the size of illumination, or combinations thereof while carrying out the present method. For example, the wavelength of a laser may be adjusted to activate different photosensitizers in the photosensitizing composition. Alternately, the power of the light source may be increased or decreased after an application of light energy to the treatment area. In addition, the light source may comprise a temperature monitoring device so that over heating of the host tissues in and around the treatment area may be avoided. Suitable temperature monitoring devices may comprise an IR device, a fiber optic device, a thermocouple, or a combination thereof.

5. Microorganisms: any and all disease-related microorganism and/or microorganisms such as virus, fungus, and bacteria. Some examples of microorganisms include but are not limited to, HIV-1, HBV, HCV, GBS, CMV, Listeria monocytogenes, Chlamydia trachomatis, E. coli, HSV, EBV, Toxoplasma gondii, HPV, and Candida.

6. Photosensitizing composition: a composition comprising at least one suitable art-disclosed photosensitizer that has at least an antimicrobial action upon application of electromagnetic energy of certain wavelength(s). Suitable photosensitizers include both Type I and Type II photosensitizers, where Type I photosensitizers produce a free radical upon the application of light and Type II photosensitizers produce singlet oxygen upon the application of light. While photosensitizers that have other modes of operation (e.g. generation of heat) are contemplated, those types discussed above are preferred. Suitable classes of compounds that may be used as antimicrobial photosensitizers include tetrapyrroles or derivatives thereof such as porphyrins, chlorins, bacteriochlorins, phthalocyanines, naphthalocyanines, texaphyrins, verdins, purpurins or pheophorbides, phenothiazines, etc., such as those described in U.S. Pat. Nos. 6,211,335; 6,583,117; and 6,607,522 and U.S. Patent Publication No. 2003-0180224. Preferred phenothiazines include methylene blue (MB), toluidine blue (TBO), and those discussed in U.S. Patent Publication No. 2004-0147508. Other preferred antimicrobial photosensitizers include indocyanine green (ICG). Combinations of two or more photosensitizers, such as MB and TBO or the like, are also suitable. The photosensitizer may be present in the photosensitizer composition in any suitable amounts. Examples are between about 0.001 percentage of total weight (wt %) and 10 wt %, between about 0.005 wt % and about 1 wt %, between about 0.01 wt % to about 0.5 wt %, and between about 0.02 wt % to about 0.1 wt %. The photosensitizing composition may optionally contain a therapeutic agent, which is any chemical, drug, medication, proteinaceous molecule, nucleic acid, lipid, antibody, antigen, hormone, nutritional supplement, cell or any combination thereof that helps ameliorate a condition. Preferred therapeutic agents include those that promote wound healing, have antimicrobial action, have anti-inflammatory action, and/or provide pain relief. The photosensitizing composition may also optionally contain carriers, diluents, or other solvents for the photosensitizer or other components of the composition and may be used to adjust the concentration of photosensitizer. The photosensitizing composition may be any suitable phase such as a liquid, gel, paste, putty, or solid. Preferably, the compositions has a viscosity low enough to flow into the treatment site while also having a viscosity high enough to maintain the composition within the treatment site. Further compositions that become liquid after application to the treatment site are contemplated such as those that melt or go into solution at the treatment site. Alternately, the composition may gel after application to the treatment site as a liquid; this would permit the composition to cover the treatment site effectively, while also maintaining the composition in the treatment site. The photosensitizers mentioned above are examples and are not intended to limit the scope of the present invention in any way.

7. Sonic energy: ultrasound, sonic waves or energy produced by a sonic or ultrasonic device. The sonic energy (e.g., vibration) is generally between the range of about 1 MHz to about 7 MHz, with exemplary ranges between about 2 KHz to about 5 MHz and between about 2 MHz to about 4 MHz.

### II. Methods to Prevent Vertical Transmission of Infectious Diseases

The present invention provides a method of preventing vertical transmission of an infectious disease comprising applying a photosensitizing composition to host tissues of a birth canal of a mother during the intrapartum period. In one embodiment, the photosensitizing composition includes the photosensitizer methylene blue at exemplary concentration ranges of from about 0.001 wt % and about 10 wt %, from about 0.01 wt % to about 5 wt %, and from about 0.1 wt % to about 1 wt %. This applying step can be accomplished with any art-disclosed applicator. It is preferred that the applicator is sterile. For example, a large sterile swab or soft foam-tipped applicator can be used.

After the photosensitizing composition has been applied to the birth canal, the method further includes applying light to the host tissues of the birth canal at a wavelength absorbed by the photosensitizing composition so as to inhibit or eliminate disease causing microorganisms that come into contact with tissues of the birth canal. It is preferred that prior to the light application step, the photosensitizing composition is placed into contact with the host tissues of the birth canal for a period of time. Examplary ranges are for at least about 1 second, at least about 5 seconds, at least about 10 seconds, at least about 30 seconds, and at least about 1 minute.

Depending on the formulation of the photosensitizing composition, the application of light can be at various wavelength(s). For example, for the photosensitizing composition containing methylene blue, the wavelength may range from about 650 nm to 685 nm, from about 660 nm to about 680 nm, and from about 665 nm to about 675 nm.

Furthermore, the amount of time desired for the light application step is generally depended upon nature of the light source and the formulation of the photosensitizing composition (e.g., the concentration of the photosensitizer(s) or the like). Examplary duration of time for the light application step ranges from about 30 seconds to about 20 minutes, from about 1 minute to about 15 minutes, and from about 3 minutes to about 10 minutes. The light energy provided during each light application step preferably ranges from about 1 J/cm² to about 25 J/cm², more preferably at about 5 J/cm² to about 20 J/Cm², and most preferably at about 6 J/cm² to about 12 J/cm².

It is preferred that during the intrapartum period, the method of applying the photosensitizing composition and light to the host tissues of the birth canal is repeated multiple times. For example, this method can be applied from about 30 minutes to every 2 hours (e.g., at least every 30 minutes, every 45 minutes, every hour, every 1½ hours, every 2 hours, etc.) during the intrapartum period. The method may optionally further include applying the photosensitizing composition and light to the host tissues of the birth canal prior to the intrapartum period. For example, a practitioner may apply the photosensitizing composition and light to the host tissues of the birth canal from at least about 5 minutes to at least about 1 hour prior to the intrapartum period.

As noted above, the practitioner may apply multiple cycles of the light application steps (e.g., about 2 to about 10, about 3 to about 5, etc.) to the host tissues of the birth canal thereby resulting in a total accumulated light energy applied to the host tissues that can be substantially higher than the light energy provided during each light application step. It is preferred that the application of light to the host tissues does not cause physiological damage to such host tissues.

The application of light can be delivered by using any suitable art-disclosed light source. For example, the light source can be the light delivery devices and systems disclosed in commonly owned co-pending U.S. patent application Ser. No. 11/741,584 titled “Photodisinfection Delivery Devices And Methods” filed on Apr. 27, 2007, which is hereby incorporated by reference.

The method of the present invention optionally further includes applying sonic energy to the host tissues of the birth canal during the intrapartum period. The sonic energy application step is achieved by applying an art-disclosed ultrasound gel either to the host tissues of the birth canal and/or the skin in the abdominal area above the birth and then applying sonic energy to the host tissues of the birth canal. The sonic energy can be supplied by any art-disclosed sonic device. For example, the sonic device can be ultrasound machines that are currently available in the market for OB/GYN uses such as the Logia models manufactured by General Electric and other models manufactured by Toshiba, Siemens, and Philips. The sonic energy (e.g., vibration) is generally preferred to be between the range of about 1 MHz to about 7 MHz, with exemplary ranges between about 2 MHz to about 5 MHz and between about 3 MHz to 4 MHz.

In one embodiment of the present invention, the applications of photosensitizing composition, light and sonic energy to the host tissues of the birth canal are repeated multiple times during the intrapartum period. For example, this method can be applied from about 30 minutes to every 2 hours (e.g., at least every 30 minutes, every 45 minutes, every hour, every 1½ hours, every 2 hours, etc.) during the intrapartum period. The method optionally includes the applications of photosensitizing composition, light and sonic energy to the host tissues of the birth canal from at least about 5 minutes to at least about 1 hour prior to the intrapartum period.

The methods described above are to be used to prevent vertical transmission of infectious diseases during the intrapartum period, especially infectious diseases caused by HIV-1, HBV, HCV, GBS, CMV, Listeria monocytogenes, Chlamydia trachomatis, E. coli, HSV, EBV, Toxoplasma gondii, HPV, and Candida.

In another embodiment of the present invention, the applications of light and sonic energy are accomplished with a vaginal probe that incorporates a suitable art-disclosed light source into a conventional vaginal ultrasound probe. Another embodiment of the vaginal probe is the light delivery device disclosed in commonly owned co-pending U.S. patent application Ser. No. 11/741,584 comprising: a member having a base portion, an insert portion adapted for insertion into a vagina of a mother and for providing desired illumination pattern for photodisinfection of host tissues of birth canal of the mother, and a pocket adapted for communication with a waveguide wherein the pocket includes a light dispersing section that is adapted for light communication with distal end of the waveguide which is connected to a light source for delivering light to the insert portion. The member further includes an art-disclosed electronic assembly adapted for providing sonic energy for sonophotodisinfection of host tissues of birth canal of the mother.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes.

What is claimed is:

1. A method of preventing vertical transmission of an infectious disease comprising:
(a) Applying a photosensitizing composition to host tissues of a birth canal of a mother during intrapartum period; and
(b) Applying light to the host tissues after the step (a) at a wavelength absorbed by the photosensitizing composition so as to inhibit or eliminate infectious disease causing microorganisms that come into contact with the host tissues.

2. The method of claim 1, the infectious disease causing microorganisms are selected from a group consisting of: human immunodeficiency virus type 1, hepatitis B virus, hepatitis C virus, Group B Streptococcus, cytomegalovirus, Listeria monocytogenes, Chlamydia trachomatis, Escherichia coli, herpes simplex virus, Epstein-Barr virus, Toxoplasma gondii, human papilloma virus and Candida.

3. The method of claim 1, wherein the step (b) takes place after the photosensitizing composition has been placed into contact with the host tissues of the birth canal for at least 30 seconds.

4. The method of claim 1, wherein duration of the step (b) is from about 3 minutes to about 10 minutes.

5 The method of claim 1, wherein the light energy provided during the step (b) ranges from about 5 J/cm² to about 20 J/cm²,

6. The method of claim 1 wherein the steps (a) and (b) are repeated at least every hour during the intrapartum period.

7. The method of claim 1 further comprising of performing the steps (a) and (b) at least about 5 minutes prior to the intrapartum period.

8. The method of claim 1 further comprising:
(c) Applying sonic energy to the host tissues after the step (b).

9. The method of claim 8 wherein the steps (a), (b) and (c) does not cause any physiological damage to the host tissues.

10. The method of claim 8 wherein the steps (a), (b) and (c) are repeated at least every hour during the intrapartum period.

11. The method of claim 8 wherein the step (c) is comprised of:
Applying ultrasound gel to the host tissues;
Applying sonic energy to the host tissues at a vibration ranging from about 2 MHz to about 5 MHz.

12. The method of claim 11 wherein the applying ultrasound gel to the host tissues step is achieved by applying the ultrasound gel onto a vaginal ultrasound probe and inserting the vaginal ultrasound probe into the birth canal.

13. The method of claim 8 wherein the light required for the step (b) and the sonic energy required for the step (c) are both provided by a vaginal probe.

14. The method of claim 13 wherein the vaginal probe is comprised of a member having a base portion, an insert portion adapted for insertion into a vagina of a mother and for providing desired illumination pattern for the step (b), and a pocket adapted for communication with a waveguide wherein the pocket includes a light dispersing section that is adapted for light communication with distal end of the waveguide which is connected to a light source for delivering light to the insert portion, and the member further includes an electronic assembly adapted for providing the sonic energy.

15. The method of claim 8, wherein the step (c) is comprised of:
Applying ultrasound gel to skin of abdominal area located above the host tissues; and
Applying sonic energy to the host tissues.

16. A method of preventing vertical transmission of human immunodeficiency virus type 1 comprising:
(a) Applying a photosensitizing composition comprising of methylene blue to host tissues of a birth canal of a mother during intrapartum period;
(b) Applying light to the host tissues after the step (a) at a wavelength from about 650 nm to about 685 nm that is absorbed by the photosensitizing composition so as to inhibit or eliminate human immunodeficiency virus type 1 that comes into contact with the host tissues; and repeating the steps (a) and (b) at least every two hour during the intrapartum period.

17. The method of claim 16 further comprising:
(c) Applying sonic energy to the birth canal after the step (b) using a sonic device.

18. The method of claim 16 wherein the method does not cause any physiological damage to the host tissues.

19. The method of claim 16, wherein the step (b) takes place after the photosensitizing composition has been placed into contact with the host tissues of the birth canal for at least 10 seconds.

20. The method of claim 16, wherein duration of the step (b) is from about 1 minute to about 15 minutes.

21. A method of preventing vertical transmission of hepatitis comprising:
(a) Applying a photosensitizing composition comprising of methylene blue to host tissues of a birth canal of a mother during intrapartum period;
(b) Applying light to the host tissues after the step (a) at a wavelength from about 660 nm to about 680 nm that is absorbed by the photosensitizing composition so as to inhibit or eliminate at least one type of hepatitis viruses that comes into contact with the host tissues; and repeating the steps (a) and (b) at least every two hour during the intrapartum period.

22. The method of claim 21, wherein the methylene blue is at a concentration range from about 0.1 wt % to about 1 wt %.

23. The method of claim 21, wherein the at least one type of hepatitis viruses is comprised of hepatitis C virus.

24. The method of claim 21, wherein the at least one type of epatitis viruses is comprised of hepatitis B virus.
